(19) United States
(12) Patent Application Publication
(10) Pub. No.: US 2024/0053623 A1
(43) Pub. Date: Feb. 15, 2024

(54) METHOD AND SYSTEM FOR DIAGNOSING THE MISMATCH OF A PART WITH A MORPHOLOGICAL CHARACTERISTIC OF A USER OF THIS PART

(71) Applicant: VIACCESS, COURBEVOIE (FR)

(72) Inventor: Pérez Pelage, Medan (FR)

(21) Appl. No.: 18/268,241

(22) PCT Filed: Dec. 7, 2021
(86) PCT No.: PCT/EP2021/084679

§ 371 Date: Jun. 18, 2023

(30) Foreign Application Priority Data
Dec. 18, 2020 (FR) 2013665

(51) Int. Cl. G02C 13/00; A61B 3/14

(52) U.S. Cl. CPC G02C 13/005; A61B 3/14

(57) ABSTRACT

This method comprises: —integrating, into an initial item, a sensor allowing measurement of a physical quantity that varies as a function both of a characteristic of the initial item and of a morphological characteristic of the user, —acquiring measurements of this sensor, to obtain a measured value, —verifying that the measured value meets a predetermined set of conditions, —when the set of conditions is not met, transmitting signal indicating mismatch of the initial term with respect to the morphological characteristic of the user, and —when the set of conditions is met, inhibiting transmission of this mismatch signal.

### CROSS-REFERENCE TO RELATED APPLICATIONS

This application is a national phase entry under 35 U.S.C. § 371 of International Patent Application PCT/EP2021/084679, filed Dec. 7, 2021, designating the United States of America and published as International Patent Publication WO 2022/128648 A1 on Jun. 23, 2022, which claims the benefit under Article 8 of the Patent Cooperation Treaty to French Patent Application Serial No. 2013665, filed Dec. 18, 2020.

### TECHNICAL FIELD

The disclosure relates to a method and system for diagnosing the mismatch of an item with respect to a morphological characteristic of a user of this item.

### BACKGROUND

The notion of morphology is here understood to include both the notion of static morphology, which is concerned with the study of forms at rest, and the notion of dynamic morphology, which is concerned with the study of forms in motion.

Such methods are notably useful for triggering corrective measures. For example, a corrective measure may be production of a new item which, this time, will be better matched to the morphological characteristics of the user. A corrective measure may also be production of a new component or of a new portion of the item. Production of a new portion of the item may, for example, consist in a repair of the item, i.e., in addition of material to the item and/or removal of material from the item. These measures may notably consist in implementation of design methods optionally followed by implementation of manufacturing methods.

These methods and systems are useful because sometimes an initial item is, from the outset, poorly matched to the morphological characteristics of the user for whom it was designed. This in particular occurs when the initial item, which is correctly shaped to fit the static morphological characteristics of the user, proves to be mismatched when the initial item is used under real-life conditions by the user, i.e., when moving about. In this text, the expression “static morphological characteristics” is understood to mean the morphological characteristics measured in a design phase of the initial item. Generally, in the design phase, these static morphological characteristics are measured while the user is at rest. This is why they are qualified “static”. However, in the design phase, it may occasionally be that a certain number of these static characteristics are measured while the user makes a predetermined movement. Specifically, under the actual conditions of use, a dynamic structural relationship between the initial item and the user may manifest itself. This dynamic structural relationship is unable to manifest itself under the design conditions of the initial item as in the design phase only static morphological characteristics are measured. This dynamic structural relationship depends on dynamic morphological characteristics of the user, which cannot, therefore, be measured under the design conditions of the initial item. In this first case, referred to as adaptation to actual conditions of use, these methods and systems are therefore used to ensure the initial item is correctly matched to the dynamic morphological characteristics of the user, which dynamic morphological characteristics cannot be measured under the design conditions of the initial item. These dynamic morphological characteristics typically result from the user moving about under the actual conditions of use of the initial item. More particularly, these dynamic morphological characteristics result from motion of the parts of the user's body that were characterized by the static morphological characteristics when the initial item was being designed. Thus, in this text, the expression “dynamic morphological characteristics” is understood to mean morphological characteristics that were not measured in the design phase of the initial item, notably because they manifest themselves when the user makes unknown movements or movements not taken into account during the design phase.

These methods and systems are also useful because the static morphological characteristics of a user may vary over time and, therefore, make an item that initially matched the morphological characteristics of this user well, mismatched as a result of the new morphological characteristics of the user. In this second case, referred to as adjustment, the methods and systems are, therefore, used to adjust the initial item to the new static morphological characteristics of the user, from those considered during design of the initial item. The case of adjustment thus includes the situation where the initial item has changed over time, or the case where the static morphological characteristics and the initial item have changed over time.

Currently, this diagnosing method is carried out by an expert in the presence of the user, under conditions of use of the item that generally reproduce only very imperfectly the actual conditions of use. Thus, detection of a mismatch of the item with respect to the morphological characteristics of the user requires the intervention of an expert. As a result, implementation of such a diagnosing method is constraining and may be complex.

One example of a method for manufacturing and verifying that spectacle lenses are correctly matched to a user's vision is described in patent application EP2899585A1. This method can be implemented only by an expert equipped with very specific measuring equipment. In addition, the specific equipment must be calibrated before each use, this making it difficult to use.

Moreover, many items worn by a user are equipped with sensors intended to measure physiological characteristics of the user. However, these known items do not make it possible to establish whether the item is correctly matched to the morphological characteristics of this user.

Examples of such items are described in WO2017/046419, FR3081565A1 or WO2016/075372A1.

### BRIEF SUMMARY

Embodiments of the disclosure aim to provide a method for diagnosing the mismatch of an item with respect to a morphological characteristic of the user, that is less constraining and simpler to implement.

One subject thereof is, therefore, such an automatic method for diagnosing the mismatch of an item with respect to a morphological characteristic of a user of this item, this method being according to claim 1.

Another subject of the disclosure is an automatic system for diagnosing the mismatch of an item according to claim 3.

### BRIEF DESCRIPTION OF THE DRAWINGS

The disclosure will be better understood on reading the following description, which is given merely by way of non-limiting example, with reference to the drawings, in which:

FIG. 1 is a schematic illustration in perspective of an item intended to be worn by a user,

FIG. 2 is a schematic illustration of a system for diagnosing the mismatch of the item of FIG. 1 with respect to the morphological characteristics of a user, and

FIG. 3 is a flowchart of a method for diagnosing the mismatch of an item with respect to the morphological characteristics of a user.

### DETAILED DESCRIPTION

In the remainder of this description, features and functions that are well known to those skilled in the art are not described in detail.

In this description, one detailed example of an embodiment is first described in section I with reference to the figures. Subsequently, in section II, variants of this embodiment are presented. Lastly, the advantages of the various embodiments are discussed in section III.

### Section I. Example of Embodiment

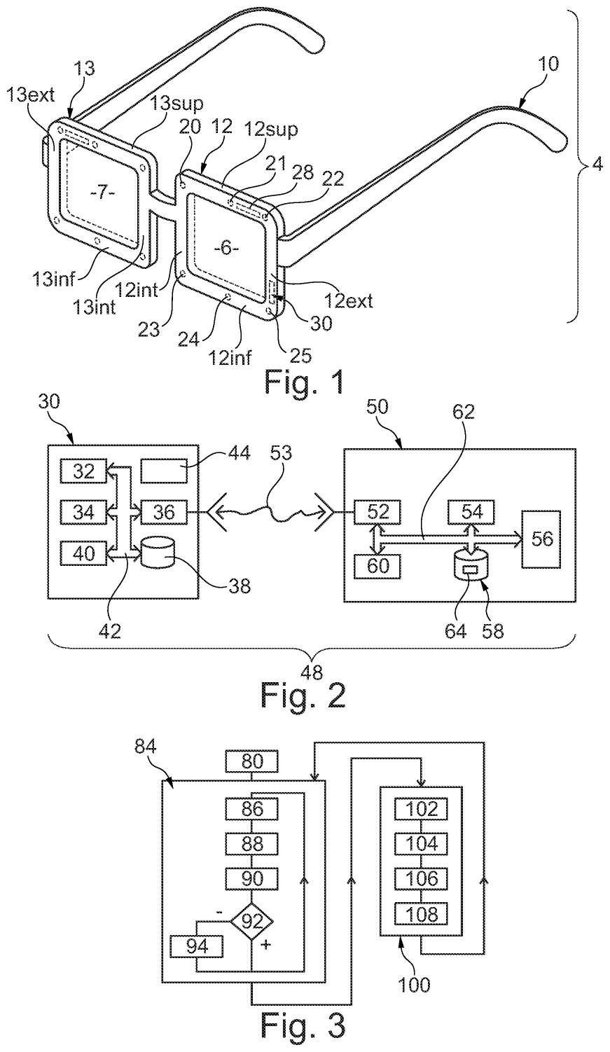

FIG. 1 shows an initial mechanical item 4 intended to be used by a user. The user is a human being. The item 4 is an item designed to specifically fit the morphological characteristics of this user. To this end, these morphological characteristics of this user were measured according to a predefined measurement protocol in an initial design phase.

Here, by way of illustration, the item 4 is a pair of glasses comprising two lenses 6 and 7 and a frame 10 to which these lenses 6 and 7 are fastened.

Here, the lenses 6 and 7 are intended to correct the user's vision. They are, therefore, specifically designed for this user. To be fully effective, these lenses must also be correctly positioned with respect to the eyes of the user.

Each of these lenses 6 and 7 mainly lies in a plane called the “median plane” below. The median plane is the one that minimizes differences, according to the least squares method, between this plane and the face of the lens closest to the user's eye in front of which the lens is located.

The frame 10 here comprises two rims 12 and 13 which encircle and grip the lenses 6 and 7, respectively. Each rim comprises:
an upper border 12sup, 13sup and a lower border 12inf, 13inf that extend mainly horizontally when the item 4 is worn by the user, and
an inner lateral border 12int, 13int and an outer lateral border 12ext, 13ext that extend mainly vertically when the item 4 is worn by the user.

The inner lateral borders 12int, 13int are the borders closest to the nose of the user when the latter is wearing the item 4. The outer lateral borders 12ext and 13ext are the borders furthest from the nose of the user when the latter is wearing the item 4.

In this embodiment, the frame 10 is a frame manufactured by 3D printing. It was specifically manufactured depending on the morphological characteristics of the user's head so that the lenses 6, 7 are correctly positioned with respect to the user's eyes. To this end, typically, static morphological characteristics of the user's head were measured, at rest, in a design phase of the item 4, according to a predefined measurement protocol that required the user's head to be in a particular position when these measurements were performed. Such a predefined protocol generally allows the repeatability of these measurements to be improved. For example, the frame 10 is formed from a single block of material.

The item 4 comprises sensors for measuring physical quantities representative of the extent to which the item 4 is matched to the morphology of the user's head. These physical quantities are representative of the static and dynamic structural relationship that exists between the item 4 and the user when the user uses this item 4. Thus, the value measured for each of these physical quantities depends both on the structural characteristics of the item 4 and on the static and dynamic morphological characteristics of the user.

In this embodiment, the physical quantities measured by the sensors of the item 4 are relative positions and orientations of certain portions of the item 4 with respect to the face of the user. These measured relative positions and orientations depend on the configuration of the item 4 and, in particular, on its dimensions. In addition, these positions and orientations also depend on the morphological characteristics of the user's face since these positions and orientations are measured with reference to these morphological characteristics. It will be recalled here that these positions and orientations may also depend on dynamic morphological characteristics of the user, which cannot be measured under the design conditions of the initial item. These dynamic morphological characteristics characterize, for example, amplitudes, angles, speeds or any other properties of the movements of the user during actual use of the item 4. In this text, the expression “actual use” is understood to mean normal use of the item 4 outside of the initial design phase. In particular, actual use is different from how the item 4 may be used in the initial design phase.

More precisely, here, the sensors of the item 4 measure physical quantities representative of the position of a lens with respect to the face of the user or to the user's eye in front of which this lens is located when the user wears the item 4. It is a question here of the following physical quantities:
physical quantities representative of the position of the optical center of the eye with respect to the edges of the lens,
   a physical quantity representative of the distance between the eye and the lens,
   a physical quantity representative of pantoscopic angle, and
   a physical quantity representative of face-form angle.

The optical center is located on a horizontal optical axis passing through the center of the pupil of the user's eye when the head of the user is vertical and she or he is looking straight ahead at the horizon. Here, the user's head is said to be vertical when she or he is holding it straight.

Here, the position of the optical center with respect to the upper or lower edge of the lens is the distance between the projection of this edge onto the median plane and the point of intersection between this median plane and the optical axis. The edge is projected onto the median plane in a direction parallel to the optical axis. Its distance is measured in the median plane when the user is wearing the item 4 and holding her or his head vertical.

Similarly, the position of the optical center with respect to the lateral edge of the lens is the distance between the projection of this lateral edge onto the median plane and the point of intersection between this median plane and the optical axis. The edge is projected onto the median plane in a direction parallel to the optical axis. In this case, the distance is measured horizontally in the median plane when the user is wearing the item 4 and holding her or his head vertical.

In the field of eyewear, pantoscopic angle is an angle that represents the inclination of the median plane when the user is wearing the item 4 and holding her or his head vertical. Thus, this pantoscopic angle substantially corresponds to the angle between this median plane and the frontal plane of the user. The frontal plane is the vertical plane that, in the reference system of anatomy, divides the user's body into an anterior or ventral portion and a posterior or dorsal portion. It is essentially parallel to the user's forehead. Pantoscopic angle depends on the configuration of the item 4 and on the morphological characteristics of the user, such as the shape of her or his nose.

Face-form angle represents the curvature, in the horizontal plane containing the optical center of the eye, of the front face of the pair of glasses when the user is wearing them. When the user is holding her or his head vertical, this face-form angle corresponds substantially to the angle between first and second straight lines. The first line is defined by the intersection between the median plane and a horizontal plane passing through the optical center. The second line is defined by the intersection between the same horizontal plane and the frontal plane of the user. Face-form angle depends on the configuration of the front face and of the temples of the item 4, and on the shape of the user's face.

In this embodiment, the sensors are fastened to the rims 12 and 13. The arrangement of the sensors fastened to the rim 13 is, typically, symmetric to the arrangement of the sensors fastened to the rim 12, with respect to the sagittal plane of the user. Thus, below, only the sensors integrated into the rim 12 are described in more detail.

The item 4 comprises a plurality of range finders fastened to the upper and lower borders 12sup, 12inf. For example, the rim 12 comprises three range finders 20 to 22 fastened to the upper border 12sup, and three range finders 23 to 25 fastened to the lower border 12inf. These range finders are fastened to the rim 12 so as to be flush with its face closest to the face of the user. These range finders are spaced apart from one another along each of the borders. In addition, the range finders 20 and 23 also belong to the inner border 12int. The range finders 22 and 25 for their part also belong to the outer lateral border 12ext. Each of these range finders 20 to 25 measures the distance between:
the point on the surface of the rim 12 with which it is flush, and
the projection of this point onto the user's face in a predetermined and fixed direction with respect to the rim 12.

For example, the predetermined and fixed direction is orthogonal to the median plane.

These range finders, in particular, make it possible to measure, when the user is wearing the item 4:
the physical quantity representative of the distance between the eye and the lens 6,
the physical quantity representative of pantoscopic angle, and
the physical quantity representative of face-form angle.

For example, the mean value of the measurements of the range finders 20 to 25 is a value representative of the distance between the lens 6 and the eye of the user. The difference between the measurements of the range finders 20 to 22 and the measurements of the range finders 23 to 25 allows a value representative of pantoscopic angle to be computed. The difference between the measurements of the range finders 20, 23 and 22, 25 allows a value representative of face-form angle to be computed.

The item 4 also comprises a camera 28 the objective of which is turned toward the eye of the user. This camera is fastened to the rim 12 so as to be flush with the face thereof closest to the face of the user. This camera 28 takes photos of the user's eye in front of which the lens 6 is located. From these photos, it is possible to extract the position of the optical center of this eye with respect to the edge of the lens 6. To do this, the position of the camera 28 with respect to the edge of the lens 6 is fixed and known. Here, the position of the optical center is considered equal to the average of the positions of the center of the pupil measured over a long period of time. For example, by “long period of time,” what is meant here is a period longer than 1 hour and, preferably, longer than one day or one week or one month. Specifically, to a first approximation, this average position of the pupil may be considered to coincide with the position of the optical center. This measured position is expressed in a reference frame tied to the frame 10. Since the position of the camera 28 and the positions of the lower, upper and lateral edges of the lens 6 are also known in this reference frame, the position of the optical center of the eye with respect to each of the edges of the lens 6 may be computed.

The item 4 also comprises a diagnostic module 30 connected to each of the sensors of the item 4. Here, the module 30 is fastened to the frame 10 and, for example, to the rim 12. The module 30 processes the sensor measurements to determine whether the item 4 is correctly matched to the morphology of the user's head. In case of mismatch, the module 30 transmits a mismatch signal. The module 30 is described in more detail with reference to FIG. 2.

FIG. 2 shows an automatic system 48 for diagnosing the mismatch of the item 4 with respect to the morphological characteristics of the user. This automatic system 48 comprises the item 4 and a device 50 for producing a new item 4. In this figure, to simplify the illustration, only the module 30 of the item 4 has been shown.

The module 30 comprises:
an interface 32 for acquiring the measurements of the various sensors of the item 4;
a human-machine interface 34, notably for transmitting a first mismatch signal perceptible by the user;
a transceiver 36 for transmitting a second mismatch signal to the device 50;
a memory 38 containing the instructions necessary to execute the method of FIG. 3;
a microprocessor 40 able to execute the instructions stored in the memory 38;
a data bus 42 that interconnects the various components of the module 30; and
a power source 44 that powers the various components of the module 30.

The human-machine interface 34 here comprises a diode that, when it is turned on, signals to the user that the item 4 is no longer matched to her or his morphological characteristics. In this case, the first mismatch signal intended for the user is emitted light.

The transceiver 36 is a local-area wireless transmitter. For example, it is a question of a Bluetooth transceiver, and preferably of a BLE transceiver (BLE standing for Bluetooth Low Energy). The transceiver 36 transmits a second mismatch signal to the device 50. This second mismatch signal is not perceptible to the user. Here, it contains the one or more measured values of the physical quantities. Just like the first mismatch signal, the second mismatch signal is transmitted by the module 30 in response to detection, by the module 30, of a mismatch of the item 4 with respect to the morphological characteristics of the user.

The memory 38 contains, in addition to the instructions of the program executed by the microprocessor 40, a predetermined set of conditions. When this predetermined set of conditions is met by the measured values of the physical quantities, the item 4 is considered to be matched to the morphological characteristics of the user. In the contrary case, the item 4 is considered to be mismatched to the morphological characteristics of the user. These conditions are notably parametrized by expected values of each of the measured values of each physical quantity. More precisely, here, it is the comparison of the measured values of the physical quantities with the expected values of these physical quantities that allows whether or not the item 4 is matched to the morphological characteristics of the user to be diagnosed.

The power source 44 typically comprises a battery capable of storing enough energy to power the various components of the module 30 for a long period of time. Preferably, this battery is rechargeable. For example, to recharge this battery, the power source 44 comprises an interface for connecting it to an external charger.

The device 50 is able to automatically produce, based on the measured values transmitted by the transceiver 36, a new item or item portion better matched to the morphological characteristics of the user. For example, here, the device 50 automatically manufactures a new frame for the user. The dimensions and configuration of this new frame are different from the dimensions and configuration of the initial frame 10, in order to obtain a better match between the morphological characteristics of the user and the new pair of glasses comprising this new frame. In this embodiment, the device 50 is typically located at a manufacturing site of the eyewear manufacturer and not at the home of the user.

To this end, the device 50 comprises:
a transceiver 52 able to set up a wireless communication link 53 with the transceiver 36, notably in order to receive the mismatch signal transmitted by the module 30;
a human-machine interface 54 allowing the measured values of the physical quantities to be displayed and production of a new frame to be triggered;
a machine 56 for automatically manufacturing spectacle frames;
a memory 58 containing the instructions necessary to execute the method of FIG. 3;
a microprocessor 60 able to execute the instructions stored in the memory 58; and
a data bus 62 for transmitting data between the various components of the device 50.

Typically, the human-machine interface 54 comprises a screen and one or more keys that are actuatable by an eyewear manufacturer.

The machine 56 is, for example, an additive manufacturing machine such as a 3D printer. Such a machine deposits successive layers of polymer or metal, for example, to manufacture the frame.

The memory 58 contains instructions for a computer-aided manufacturing module 64. This computer-aided manufacturing module 64 notably comprises a digital model of the model of the frame 10, the digital model being parametrized by the differences between the measured values of the physical quantities and the expected values of these physical quantities. The expected values are predetermined constants. The expected values are predetermined independently of the morphological characteristics of a particular user. They are target values that when reached mean that the item 4 may be considered correctly matched to its user. These target values are are, for example, chosen by the eyewear manufacturer or set by the manufacturer of the lenses or by any standard applicable in the art. Thus, as soon as the measured values of these physical quantities are known, all the dimensions of the new frame to be produced are known. Based on this model parametrized with the measured values of the various physical quantities, the computer-aided manufacturing module 64 is able, when it is executed by the microprocessor 60, to generate a command file executable by the machine 56. When the machine 56 executes this command file, it automatically manufactures a frame the configuration and dimensions of which are identical to those of the model parametrized by the measured values of the physical quantities.

The operation of the automatic system 48 will now be described with reference to the method of FIG. 3.

The method begins with a phase 80 of producing the initial item 4. In this phase 80, the initial item 4 is manufactured. For example, the user chooses, among a high number of frame models, the model that she or he likes. Next, the chosen frame model is produced by the device 50, so as to obtain the frame 10. To do this, the device 50 receives, via the human-machine interface 54, values, measured at rest, of the static morphological characteristics of the user and an identifier of the chosen frame model. The measurements of the static morphological characteristics were typically made in a preliminary design phase of the initial item 4 under conditions termed the design conditions of the initial item 4. Based on these entered measurements, on the expected values of the various physical quantities and on the digital model of the chosen frame model, the device 50 automatically establishes the dimensions of a frame that will allow the expected values of the physical quantities to be obtained when this frame is worn by this user. Next, the microprocessor executes the computer-aided manufacturing module 64 to generate the command file for the machine 56. The eyewear manufacturer may then trigger manufacture of the corresponding frame 10.

The machine 56 then manufactures the frame 10. Once the frame 10 has been manufactured, the eyewear manufacturer permanently assembles the lenses 6 and 7, the various sensors and the diagnostic module 30 with the frame 10 and obtains the initial item 4. The expression “permanently assemble” is here understood to mean that the lenses 6, 7, the sensors and the diagnostic module 30 remain fastened to the frame 10 during actual use of this frame 10. In other words, this permanent assembly ensures that the lenses 6, 7, the sensors and the diagnostic module 30 are worn by the user each time the frame 10 is worn by this user. However, this does not prevent these elements from being dismounted if necessary and without damaging the item 4, for example, so as to be replaced. The expected values of each of the physical quantities measured using the sensors of the item 4 will be stored in the memory 38.

A predetermined set of conditions on the measured values of the physical quantities is also stored in the memory 38. This set of conditions notably includes conditions on the difference between the measured value and the expected value of each physical quantity. These conditions are met if the difference between the measured value and the expected value of each physical quantity remains comprised between a predetermined threshold Sₘᵢₙ and a predetermined threshold Sₘₐₓ. As soon as the difference crosses one of these thresholds Sₘᵢₙ and Sₘₐₓ, the condition is no longer met and, therefore, the set of conditions is no longer met.

The expected values and the predetermined set of conditions are stored in the memory 38, for example, by the device 50 and via the wireless communication link 53.

The initial item 4 thus produced is then given to the user. A phase of actual use of this item 4 by the user then begins. This phase of actual use lasts a long period of time. The phase of actual use comprises periods of use of the item 4 and periods of absence of use of the item 4. During the periods of use, the item 4 is continuously used and worn by the user. During periods of absence of use, the item 4 is not used and is not worn by the user.

During the phase of actual use of the item 4, a phase 84 of verifying the match of this item 4 with respect to the morphological characteristics of the user is executed. The duration of this verifying phase 84 is tailored to the type of mismatch to be detected. In the first case of adaptation to the actual conditions of use, the duration of phase 84 is preferably short with respect to the expected lifespan of the initial item 4. In this first case, the duration of phase 84 is typically of the order of a few thousandths to a few hundredths of the expected lifespan of the initial item 4, and, for example, comprised between 0.4% and 5% of this expected lifespan. In the second case of adjustment, the duration of phase 84 may be long with respect to the expected lifespan of the initial item 4, typically of the same order of magnitude as this expected lifespan, and, for example, longer than 25%, 50%, 100%, or 150% of this expected lifespan.

More precisely, in step 86, the sensors integrated into the frame 10 each perform their own measurement.

In step 88, the module 30 acquires the measurements taken by the sensors of the frame 10.

Each time new measurements are acquired, in step 90, the module 30 processes these measurements. This processing here consists in executing, in order, the following two operations:
1) detecting a period of use of the item 4 and, otherwise, detecting a period of absence of use of the item 4, and
2) only if a period of use is detected, computing, based on the individual measurements of the various sensors, a measured value of each of the physical quantities of interest.

Here, a period of absence of use is detected using the same sensors as those used to establish the measured value of each of the physical quantities of interest. There are many different ways to detect a period of absence of use based on the measurements of the sensors (e.g., range finders 20 to 25) and/or based on the image taken by the camera 28. For example, the following embodiments are possible:
First embodiment: The module 30 seeks to identify the presence of an eye in the image taken by the camera 28. To do this, the module 30 searches in the image for a pattern characteristic of the presence of an eye. For example, this pattern may be a pupil, the white of the eye, an eyebrow, inter alia. If this search is unsuccessful, a period of absence of use is detected. In the contrary case, a period of use is detected.
Second embodiment: The module 30 records the measurements of the range finders 20 to 25 and compares the measured distances to a threshold S_{useful}. The value of the threshold S_{useful} is chosen so that this threshold is exceeded only when the item 4 is not being worn. If the distance measured by most of the range finders 20 to 25 is larger than this threshold S_{useful}, a period of absence of use is detected. In the contrary case, a period of use is detected.

If a period of absence of use is detected in step 90, the method immediately returns to step 86 without executing the following steps.

If a period of use was detected in step 90, then, in step 92, the module 30 verifies whether the set of conditions is met by the measured values of the physical quantities.

In step 94, if the set of conditions is not met, the module 30 transmits a mismatch signal. The mismatch thus flagged may be due to an original mismatch with respect to the actual conditions of use or to the appearance of a need to adjust the item 4 to the user's morphological characteristics considered in their entirety.

Here, if the item 4 is too far from the device 50 to set up the wireless communication link 53, this mismatch signal is transmitted via the human-machine interface 34. Thus, initially, when the item 4 is no longer matched to the morphological characteristics of the user, the user is informed of this via this human-machine interface 34. Next, the method returns to step 86. Thus, as long as the predetermined set of conditions is not met, step 94 is executed.

If the predetermined set of conditions is met, the method returns to step 86 without executing step 94. Transmission of the mismatch signal is then inhibited.

Step 86 and the following steps are reiterated at a regular interval at least for a cumulative time DC of use. The time DC is equal to the sum of the durations of a succession of consecutive periods of use.

In the case where the mismatch to be flagged is an original mismatch with respect to the actual conditions of use, the duration of the regular interval is shorter than five minutes or than one minute and the time DC is longer than 48 hours or 72 hours or 300 hours. The duration of the regular interval is generally longer than one second or ten seconds. In this case, beyond the time DC, execution of phase 84 may be systematically interrupted.

In the case where the mismatch to be flagged is a need to adjust the item 4, the duration of the regular interval is much longer. For example, the duration of the regular interval is longer than one day or one week or one month or one year. The cumulative time DC is also much longer. For example, in this case, the time DC is longer than one, three, or six months, longer than one year, or longer than two years, or longer than five years.

When the user is informed of the mismatch of the item 4 to her or his own morphological characteristics, she or he goes to an eyewear manufacturer with her or his item 4.

A phase 100 of producing a new item 4 may then begin.

At the start of this phase 100, in step 102, the wireless communication link 53 between the module 30 of the item 4 and the device 50 is set up.

In response, in step 104, the module 30 transmits a mismatch signal to the device 50 via this wireless communication link 53. The mismatch signal transmitted to the device 50 comprises the measured values of the physical quantities that caused transmission of this mismatch signal to be triggered. These measured values are then acquired by the microprocessor 60.

In step 106, the microprocessor 60 replaces, in the digital model of the frame model chosen by the user in phase 80, the parameters that correspond to the measured physical quantities with the measured values acquired in step 104. Next, the computer-aided manufacturing module 64 is executed, this converting the model parametrized by the acquired values into a command file. This command file is then transmitted to the machine 56.

In step 108, the machine 56 executes the new command file received. This leads the machine 56 to manufacture a new frame sized and shaped depending on the measured values acquired in step 104. This new frame is specifically sized and shaped so that, when it is worn by the user, the predetermined set of conditions is once again met.

As in phase 80, once the new frame has been manufactured, the eyewear manufacturer permanently assembles the lenses 6 and 7, the various sensors and the module 30 with this new frame. During production of the new item 4, generally, the expected values and the set of conditions stored in the memory 38 are the same as those stored in this memory 38 in the initial item 4, because the frame model is the same.

The eyewear manufacturer thus obtains a new item 4 which she or he gives to the user. Phase 100 ends and anew phase of actual use begins. In this new phase of actual use, phase 84 is once again executed, but this time with the new item 4 and not with the initial item 4.

### Section II. Variants

Variants of the Diagnostic Module:

As a variant, the diagnostic module 30 is not incorporated into the item 4. For example, the module 30 is incorporated into the device 50 for producing the new item 4. In this case, the sensor measurements are stored in the memory 38 and transmitted to the diagnostic module 30 via the wireless communication link 53. Consequently, for a mismatch signal to be transmitted, the user must take the item 4 to an area where transmission of the sensor measurements to the module 30 integrated into the device 50 is possible. In another embodiment, the sensors of the item 4 transmit their measurements to the user's mobile telephone as they are taken. In response, the mobile telephone sends these measurements to the diagnostic module 30 integrated in the device 50 via a wide-area data-transmitting network, such as a cellular network.

Other embodiments of the predetermined set of conditions are possible. For example, the set of conditions comprises alternative conditions. Thus, the set of conditions is met provided that at least one of the alternative conditions is met.

Transmission of the mismatch signal from the module 30 to the device 50 may occur via any data-transmitting network and not necessarily via a local-area wireless link. In particular, it may be transmitted via a wide-area data-transmitting network, such as, for example, a cellular network. For example, the module 30 integrated into the item 4 transmits the mismatch signal to the user's mobile telephone. Next, the mobile telephone relays the mismatch signal to the device 50 via a cellular network.

In one simplified embodiment, the mismatch signal does not comprise the measured values of the physical quantities. For example, the mismatch signal is a simple auditory or visual or tactile signal perceptible by the user. In response to receipt of the mismatch signal thereby, the user may then make an appointment with the eyewear manufacturer. Alternatively, in response to perception of this signal, the user may be required to go to the eyewear manufacturer, who will then once more take the measurements required to produce a new item 4 matched to the morphological characteristics of the user. In the latter case, the module 30 is not necessarily capable of sending the measured values of the physical quantities to an external device such as the device 50.

In another embodiment, the human-machine interface 34 comprises a button that, in response to being pressed, systematically triggers transmission, by the transceiver 36, of the measured values of the physical quantities. In this case, the measured values are not transmitted solely in response to detection of a mismatch between the item and the morphological characteristics of the user.

As a variant, the human-machine interface 34 is omitted. In this case, it may be replaced by a human-machine interface mechanically independent of the item 4. For example, the human-machine interface is integrated into the cellphone of the user. In another variant, the mismatch signal is only transmitted to the device 50. In the latter case, it is the human-machine interface 54 or the production of a new item that informs the user that the initial item 4 is no longer matched to her or his morphology.

Other embodiments of the human-machine interface 34 are possible. For example, the human-machine interface 34 may comprise instead of, or in addition to, the diode:
a loudspeaker for generating a sound that signals to the user that the item 4 is no longer matched, and/or
a vibrator that generates vibrations perceptible to the user to inform her or him that the item 4 is no longer matched.

Other embodiments of the power source 44 are possible. For example, the power source 44 comprises a battery charger capable of charging the battery by harvesting energy present in the external environment of the item 4. For example, the power source 44 comprises a photovoltaic panel or a transducer that converts movements into electrical energy.

Variants of the Device 50:

As a variant, the item 4 comprises means for adjusting its configuration. For example, in the case of a pair of glasses, this pair of glasses comprises screws that allow the spacing between the temples of this pair of glasses to be adjusted. In this case, production of the new item may simply consist in adjusting these adjusting means. After having adjusted these adjusting means, the item obtained is new because it has a different configuration from the initial item. For example, the spacing between the temples of the pair of glasses is different from that observed in the initial pair of glasses. Thus, production of a new item does not necessarily imply implementation of steps of manufacturing one portion or the entirety of this item. When such adjustments to the configuration of the item 4 are able to be made by hand by a human being, the device 50 may be omitted or replaced by a human-machine interface that simply displays the measured values of the physical quantities and that does not comprise any manufacturing machine such as the machine 56.

Production of the new item may also consist in adding material to the initial item and/or removing material from the initial item.

The device 50 may also be an automatic device for adjusting the item 4, able to automatically adjust these adjusting means.

In another embodiment, the new item differs from the initial item not in its dimensions but in the material from which the new item is made. For example, the new item produced is identical to the initial item except that it is made from a material having different mechanical properties. For example, it is made from a material having a Young's modulus lower than 0.8Yini or higher than 1.2Yini, where Yini is the value of the Young's modulus of the material used to produce the initial item.

As a variant, the device 50 is replaced or supplemented by a machine for machining corrective lenses. In this case, in response to receipt of a mismatch signal, the device 50 automatically manufactures new lenses for the user.

Other embodiments of the machine 56 are possible. For example, the machine 56 is able to manufacture the frame 10 but is a computer-aided subtractive manufacturing machine. For example, as a variant, the machine 56 is replaced or supplemented by a machine tool, a machine for injecting plastic or carrying out injection molding, an assembling machine, or the like. More generally, the machine 56 may be any Industry 4.0 manufacturing machine or any computer-aided manufacturing machine. In another embodiment, the frame 10 is manufactured by a manufacturing machine that is not computer aided.

The device 50 is able to produce at least one portion of the new item. Thus, depending on the portion of the item or the item to be manufactured, the machine 56 may be very different from the one described with reference to the figures. However, the teaching given here in the particular case of a frame of a pair of glasses may be transposed without particular difficulty to items other than a pair of glasses or to portions of items other than a pair of glasses. In particular, what has been described so far in the particular case where the item is a pair of glasses applies to other items intended to be worn by the user. For example, the item may also be chosen from the group consisting of:
a pair of binoculars, an ocular view-finding device of a still camera, of a video camera or of a weapon,
a helmet, a hat, an item of clothing such as a glove, a pair of trousers or a jacket, a shoe,
a hearing aid or a prosthesis,
a handle of an item of equipment such as a still camera, a racket, a tool or a weapon.

Variants of the Method:

The device that makes the initial item is not necessarily the same as the device that makes the new item. As a variant, it may be a question of two different devices.

Assembly of the lenses 6 and 7 and/or sensors may also be automated and, therefore, carried out automatically by a machine.

As a variant, the predetermined set of conditions is stored in the memory 38 using a programming unit of the module 30 that is independent of the device 50.

There are other ways of detecting the periods of use and of absence of use based on the measurements of the sensors of the item 4. For example, only the distance measured by the range finder 20 or 23 is compared to the threshold S_{useful}. If the distance measured by this range finder 20 or 23 exceeds this threshold, then a period of absence of use is detected. In another embodiment, it is the average of the distances measured by the range finders 20 to 25 that is compared with the threshold S_{useful}. In certain embodiments, when the distance which separates a range finder from its target is larger than the threshold S_{useful}, the range finder delivers an identifier that indicates that the target is too far away for a distance measurement to be performed. In this case, it is the presence of this identifier that is used to detect a period of absence of use.

In one simplified embodiment, the detection of periods of use and of absence of use is omitted. This is, for example, the case if the item is an item, such as a prosthesis, permanently worn by the user during the phase of actual use.

The mismatch signal and the measured values of the physical quantities that triggered its transmission may be stored in the memory 38 at a time when the user is wearing the item 4. Then, subsequently, the stored mismatch signal is transmitted to the device 50. Thus, the presence of the user in phase 100 is not necessary.

There are other ways of making the new item. For example, as a variant, the new item does not comprise the sensors or the diagnostic module 30. In this case, after phase 100, the method does not return to phase 84 but continues with an ordinary phase of use of the new item 4 without the possibility of detecting its mismatch to the morphological characteristics of the user.

Other Variants:

The sensors may be integrated into the item 4 in another way. For example, as a variant, the camera 28 is fastened to the lens 6 and not to the frame 10. Likewise, the range finders 20 to 25 may also be fastened directly to the lens 6 and not to the frame 10.

The sensors may also be integrated into the item 4 in such a way that these sensors are not removable. In this case, it is not possible to disassemble the sensors without damaging the item 4.

As a variant, the human-machine interface 34 is able to acquire a command from the user to trigger the phase 84 of verifying the match of the item 4 to her or his morphological characteristics. For example, to this end, the human-machine interface 34 comprises a button. When this button is pressed, the module 30 executes phase 84. When the button is released, execution of phase 84 stops. In this case, detection of the periods of use and of absence of use may be omitted. To increase how quickly the position of the optical center of the eye with respect to the edges of the lens is determined, after execution of phase 84 has been triggered, the user stands straight and gazes forward fixedly at the horizon until she or he ends execution of this phase 84 by releasing the button. To help the user to direct her or his gaze in a predetermined direction, an external apparatus independent of the item 4 may be used to display a target that the user must look at.

The physical quantity representative of the mismatch of the item with respect to the morphological characteristics of the user depends on the item. For example, the measured physical quantity may be chosen from the group consisting of the following physical quantities:
the position and/or orientation of the item with respect to the user's body;
the pressure exerted by the item on the body of the user;
the temperature of the user's body at a point of contact with the item;
vibrations generated by the item rubbing against the user's skin;
a variation in the color of the skin generated by the item rubbing against this skin.

For example, a pressure sensor may be embedded in one of the temples of the pair of glasses to measure the pressure exerted by this temple on the user's head.

A temperature, vibration or color sensor may be integrated into an item such as a shoe to measure a physical quantity representative of rubbing between the skin and the shoe.

Specifically, this rubbing produces heat and vibrations.

The one or more sensors used to detect the periods of use and of absence of use of the item 4 are not necessarily the same as those that measure the quantity of interest. For example, the item 4 may comprise an additional sensor only used to detect the periods of use and of absence of use. For example, in the case where the item 4 is a pair of glasses, this additional sensor may be the pressure sensor described above or a temperature sensor. In the case where such an additional sensor is used, the performance of measurements, by the other sensors that measure the physical quantity of interest, may be inhibited for the entire length of each period of absence of use.

The initial morphological characteristics may be morphological characteristics measured on the user, for example by means of an application of a mobile terminal of the user such as a smartphone or a tablet, and transmitted by the user, or pre-recorded standard morphological characteristics assumed to be suitable for all users. Thus, the initial morphological characteristics used to manufacture the initial item 4 are not necessarily measured on the user.

What has been described here in the particular case where the user is a human being also applies to cases where the user is a living being such as an animal.

### Section III: Advantages of the Described Embodiments

The method described here makes it possible to automatically flag a mismatch of an item with respect to the morphological characteristics of the user for whom it was produced. This is possible without the intervention or presence of an expert. Therefore, the intervention of a person with the appropriate expertise to detect such mismatch is no longer necessary. This, therefore, greatly simplifies implementation of such a diagnostic method.

Integrating the sensors into the item allows, during each measurement sequence, the sensors to be kept in the same position with respect to the user. Thus, prior to each measurement sequence, it is not necessary to carry out a phase of calibrating the measurements.

In addition, integrating the sensors into the item 4 allows the measurements to be performed during actual use of the item. This, therefore, makes it possible to detect a mismatch of the item caused by the fact that dynamic morphological characteristics might not have been taken into account during the initial design of the item.

In a phase of actual use of the item 4, the latter is generally not constantly used by the user. Detecting periods of absence of use of the item 4, therefore, makes it possible to take into account, to establish the diagnosis, only measured values constructed from measurements performed during the periods of use of the item 4 by the user. This improves the reliability of the diagnosis.

Repeating the performance of the measurements at regular intervals of duration shorter than five minutes and for a time of use longer than 48 hours, allows a mismatch of the item 4 caused by a dynamic morphological characteristic that exists only at certain times during the actual use of the item 4 to be detected. For example, this makes it possible to detect a mismatch of the item 4 during the practice of a physical activity, such as a sport, at a certain time of the day or on certain days.

1. An automatic method for diagnosing the mismatch of an item with respect to a morphological characteristic of a user of this item, this method comprising:
producing an initial item shaped, depending on a predetermined morphological characteristic of a user of this item, to obtain a predetermined relationship between this initial item and the user having this morphological characteristic, this predetermined relationship corresponding to an expected value of a physical quantity that depends both on a characteristic of the item and on the morphological characteristic of the user,
using at least one sensor allowing measurement of the physical quantity that varies as a function both of the characteristic of the initial item and of the morphological characteristic of the user,
performing measurements by means of the sensor,
acquiring and processing these measurements by means of an automatic diagnostic module, to obtain a measured value of the physical quantity,
verifying, by means of the diagnostic module, that the measured value meets a predetermined set of conditions, this set of conditions being parametrized by the expected value of the physical quantity,
when the set of conditions is not met, transmitting, by means of the diagnostic module, a signal indicating mismatch of the initial item with respect to the morphological characteristic of the user, and
when the set of conditions is met, inhibiting, by means of the diagnostic module, transmission of this signal indicating mismatch of the initial item with respect to the morphological characteristic of the user,
wherein the method comprises permanently integrating, into the produced initial item, said at least one sensor allowing measurement of the physical quantity that varies as a function both of the characteristic of the initial item and of the morphological characteristic of the user.

2. The method as claimed in claim 1, wherein the method comprises:
transmitting the measured value that triggered transmission of the mismatch signal, then
acquiring the measured value transmitted by an automatic device for producing a new item, then
producing, by means of this automatic device, the new item shaped, this time, depending on the acquired measured value, to obtain the predetermined relationship between this new item and the user when this new item is used by the user.

3. The method as claimed in claim 1, wherein:
the method comprises detecting a period of use and, alternately, a period of absence of use, a period of use being a period during which the initial item is worn continuously by the user and a period of absence of use being a period during which the initial item is not worn by the user, and
in response to detection of a period of absence of use, performance of measurements by the sensor is inhibited throughout this period of absence of use or the verification, by the diagnostic module, that the measured value meets a predetermined set of conditions is carried out taking into account only measurements taken by the sensor outside of this period of absence of use.

4. The method as claimed in claim 1, wherein measurement is performed, by the sensor, repeatedly at a regular interval for a time longer than 48 hours and the duration of this regular interval is shorter than five minutes.

5. An automatic system for diagnosing the mismatch of an item with respect to a morphological characteristic of a user of this item, this system comprising:
an initial item shaped, depending on a predetermined morphological characteristic of a user of this item, to obtain a predetermined relationship between this initial item and the user having this morphological characteristic, this predetermined relationship corresponding to an expected value of a physical quantity that depends both on a characteristic of the item and on the morphological characteristic of the user,
a sensor able to perform measurements,
an automatic diagnostic module configured to:
acquire and process the sensor measurements to obtain a measured value of the physical quantity,
verify that the measured value meets a predetermined set of conditions, this set of conditions being parametrized by the expected value of the physical quantity,
when the set of conditions is not met, transmit a signal indicating mismatch of the initial item with respect to the morphological characteristic of the user, and
when the set of conditions is met, inhibit transmission of this signal indicating mismatch of the initial item with respect to the morphological characteristic of the user,
wherein the sensor is integrated, permanently, into the produced initial item.

6. The system as claimed in claim 5, wherein:
the diagnostic module is configured to transmit the measured value that triggered transmission of the mismatch signal, and
the system comprises an automatic device for producing a new item, this device being able:
to acquire the measured value transmitted by the diagnostic module, then
to produce the new item shaped, this time, depending on the acquired measured value, to obtain the predetermined relationship between this new item and the user when this new item is used by the user.

7. The system as claimed in claim 5, wherein the characteristic of the item is chosen from the group consisting of:
the dimensions of the item, and
the material from which the item is produced.

8. The system as claimed in claim 5, wherein the physical quantity is the relative position or the relative orientation of the item with respect to a part of the body of the user.

9. The system as claimed in claim 8, wherein:
the initial item and the new item are pairs of glasses comprising lenses intended to be positioned in front of a respective eye of the user, and
the physical quantity is chosen from the group consisting of the following physical quantities:
a physical quantity representative of the position of the optical center of the user's eye with respect to an edge of the lens located in front of this eye when the user is wearing this pair of glasses,
a physical quantity representative of the distance between the eye and the lens located in front of this eye when the user is wearing this pair of glasses,
a physical quantity representative of pantoscopic angle when the user is wearing this pair of glasses,
a physical quantity representative of face-form angle when the user is wearing this pair of glasses.

10. The the system as claimed in claim 9, wherein:
the diagnostic module is configured to transmit the measured value that triggered transmission of the mismatch signal,
the system comprises an automatic device for producing a new item, this device being able:
to acquire the measured value transmitted by the diagnostic module, then
to produce the new item shaped, this time, depending on the acquired measured value, to obtain the predetermined relationship between this new item and the user when this new item is used by the user; and
the automatic device is able, in response to acquisition of the measured value, to:
produce a new lens for a pair of glasses depending on the acquired measured value, and/or
produce a new frame for a pair of glasses depending on the acquired measured value.

11. The system as claimed in claim 5, wherein the set of conditions comprises at least one condition that compares, with a predetermined threshold, the difference between the measured value and the expected value of the physical quantity, this condition being met only if the difference is less than this predetermined threshold.

12. The method as claimed in claim 1, wherein the characteristic of the item is chosen from the group consisting of:
the dimensions of the item, and
the material from which the item is produced.

13. The method as claimed in claim 1, wherein the physical quantity is the relative position or the relative orientation of the item with respect to a part of the body of the user.

14. The method as claimed in claim 13, wherein:
the initial item and the new item are pairs of glasses comprising lenses intended to be positioned in front of a respective eye of the user, and
the physical quantity is chosen from the group consisting of the following physical quantities:
a physical quantity representative of the position of the optical center of the user's eye with respect to an edge of the lens located in front of this eye when the user is wearing this pair of glasses,
a physical quantity representative of the distance between the eye and the lens located in front of this eye when the user is wearing this pair of glasses,
a physical quantity representative of pantoscopic angle when the user is wearing this pair of glasses,
a physical quantity representative of face-form angle when the user is wearing this pair of glasses.

15. The method as claimed in claim 14, wherein:
the method comprises:
transmitting the measured value that triggered transmission of the mismatch signal, then
acquiring the measured value transmitted by an automatic device for producing a new item, then
producing, by means of this automatic device, the new item shaped, this time, depending on the acquired measured value, to obtain the predetermined relationship between this new item and the user when this new item is used by the user, and
the automatic device is able, in response to acquisition of the measured value, to:
produce a new lens for a pair of glasses depending on the acquired measured value, and/or
produce a new frame for a pair of glasses depending on the acquired measured value.

16. The method as claimed in claim 1, wherein the set of conditions comprises at least one condition that compares, with a predetermined threshold, the difference between the measured value and the expected value of the physical quantity, this condition being met only if the difference is less than this predetermined threshold.
